(19) United States
(12) Patent Application Publication
(10) Pub. No.: US 2022/0365068 A1
(43) Pub. Date: Nov. 17, 2022

(54) ALCOHOL DETECTOR, INFORMATION PROCESSING DEVICE, STORAGE MEDIUM, AND ALCOHOL DETECTION SYSTEM

(71) Applicant: PAI-R Co., Ltd., Osaka (JP)

(72) Inventors: Isao YASUDA, Osaka (JP); Hiroshi KUZE, Osaka (JP)

(21) Appl. No.: 17/746,166

(22) Filed: May 17, 2022

(30) Foreign Application Priority Data
May 17, 2021 (JP) 2021-083224

(51) Int. Cl. G01N 33/497

(52) U.S. Cl. CPC G01N 33/4972

(57) ABSTRACT

An alcohol detector includes a detecting section, a display section, a communication section, and a controller. The detecting section performs alcohol concentration detection in exhaled breath blown by a subject. The communication section communicates with a prescribed external device performing image capture of the subject who blows the exhaled breath. The controller causes the detecting section to perform the alcohol concentration detection and transmits a detection result of the alcohol concentration detection to the prescribed external device via the communication section. The controller causes the display section to display an image including identification information indicating the alcohol detector during image capture of the subject in a time from a start of the alcohol concentration detection to transmission of the detection result.

### INCORPORATION BY REFERENCE

The present application claims priority under 35 U.S.C. § 119, based on Japanese Patent Application No. 2021-83224 filed on May 17, 2021. The contents of this application are incorporated herein by reference in their entirety.

### BACKGROUND

The present disclosure relates to an alcohol detector, an information processing device, a storage medium and an alcohol detection system.

Patent Literature 1 (Japanese Patent Application Laid-Open Publication No. 2015-175665) discloses a technique for preventing deception in an alcohol detection using an alcohol detector. Specifically, an exhalation component measuring system described in Patent Literature 1 includes an exhalation component measuring device and an information processing terminal communicatively connected to the exhalation component measuring device. The exhalation component measuring system captures an image including a face of a subject with an imaging device of the information processing terminal when the subject puts a mouthpiece of the exhalation component measuring device into the mouth. After image capturing, the exhalation component measuring device urges the subject to blow into the mouthpiece within a predetermined time (e.g., 5 seconds). If the subject has not breathed air into the exhalation component measuring system within the predetermined time after the image capturing, the exhalation component measuring system restarts from image capturing. Therefore, it is difficult to change the subject after imaging.

### SUMMARY

The alcohol detector exemplified in the present disclosure preferably includes a detecting section, a display section, a communication section, and a controller. The detecting section preferably performs alcohol concentration detection in exhaled breath blown by a subject. The communication section preferably communicates with a prescribed external device performing image capture of the subject who blows the exhaled breath. The controller preferably causes the detecting section to perform alcohol concentration detection and transmits a detection result of the alcohol concentration detection to the prescribed external device via the communication section. The controller preferably causes the display section to display an image indicating identification information indicating the alcohol detector during the image capture of the subject in a time from a start of the alcohol concentration detection to transmission of the detection result.

The information processing device exemplified in the present disclosure preferably includes a communication section, an imaging section, and a controller. The communication section preferably communicates with an alcohol detector that performs alcohol concentration detection of exhaled breath of a subject, displays an image including identification information indicating the alcohol detector, and outputs a detection result of the alcohol concentration detection. The controller preferably: causes the imaging section to perform image capture during display of an image including at least the identification information before the detection result is output by the alcohol detector; acquires the detection result of the alcohol concentration detection via the communication section after the imaging section starts the image capture; and determines whether or not the acquired detection result is valid based on the identification information and a captured image captured by the imaging section. In determining whether or not the detection result is valid, the controller determines that the detection result is invalid when the captured image does not include an image matching the identification information.

An exemplary non-temporary computer-readable storage medium of the present disclosure preferably stores a program that causes an information processing device to execute processing. The information processing device includes an imaging section and a communication section that communicates with an alcohol detector. The alcohol detector performs alcohol concentration detection of exhaled breath of a subject, displays an image including identification information indicating the alcohol detector, and outputs a detection result of the alcohol concentration detection. The above-mentioned processing preferably includes: causing the imaging section to perform image capture during display of an image including at least the identification information before the detection result is output by the alcohol detector; acquiring the detection result of the alcohol concentration detection via the communication section after the imaging section starts the image capture; and determining whether or not the acquired detection result is valid based on the identification information and a captured image captured by the imaging section. In determining whether or not the acquired detection result is valid, it is preferably determined that the detection result is invalid when the captured image does not include an image matching the identification information.

An exemplary alcohol detection system of the present disclosure preferably includes an alcohol detector and an information processing device communicatively connected to each other. The alcohol detector preferably includes a detecting section, a display section, and a first controller. The detecting section preferably performs alcohol concentration detection in exhaled breath blown by a subject. The first controller preferably causes the detecting section to perform the alcohol concentration detection and transmits a detection result of the alcohol concentration detection to the information processing device. The first controller preferably causes the display section to display an image indicating identification information indicating the alcohol detector during a time from a start of the alcohol concentration detection to transmission of the detection result. The information processing device preferably comprises an imaging section and a second controller. The second controller preferably: causes the imaging section to perform image capture during display of an image including at least the identification information before the detection result is output by the alcohol detector; acquires the detection result of the alcohol concentration detection after the image section starts image capture; and determines whether or not the acquired detection result is valid based on the identification information and a captured image captured by the imaging section. In determining whether or not the acquired detection result is valid, the second controller preferably determines that the detection result is invalid when the captured image does not include an image matching the identification information.

### BRIEF DESCRIPTION OF THE DRAWINGS

FIG. 1 is a schematic diagram illustrating a schematic configuration of an alcohol detection system according to a first embodiment.

FIG. 2 is a block diagram illustrating a schematic configuration of an alcohol detector illustrated in FIG. 1.

FIG. 3 is a block diagram illustrating a schematic configuration of an information processing device illustrated in FIG. 1.

FIG. 4A is a flowchart illustrating an operation of the alcohol detector according to the first embodiment.

FIG. 4B is a flowchart illustrating an operation example of the information processing device according to the first embodiment.

FIG. 5 is a schematic diagram illustrating a situation in which a subject performs alcohol detection.

FIG. 6A is a flowchart illustrating an operation example of an alcohol detector according to a second embodiment.

FIG. 6B is a flowchart illustrating an operation example of an information processing device according to the second embodiment.

FIG. 7 is a flowchart illustrating an operation example of an alcohol detector according to a third embodiment.

FIG. 8 is a block diagram illustrating a schematic configuration of an alcohol detector and an information processing device according to a fourth embodiment.

FIG. 9A is a flowchart illustrating an operation example of the alcohol detector according to the fourth embodiment.

FIG. 9B is a flowchart illustrating an operation example of the information processing device according to the fourth embodiment.

FIG. 10 is a block diagram illustrating a schematic configuration of an alcohol detector and an information processing device according to a fifth embodiment.

FIG. 11A is a flowchart illustrating an operation example of the alcohol detector according to the fifth embodiment.

FIG. 11B is a flowchart illustrating an operation example of the information processing device according to the fifth embodiment.

### DETAILED DESCRIPTION

The following describes embodiments of the present disclosure with reference to the accompanying drawings. In the drawings, like elements will be indicated using like reference signs and descriptions thereof are not repeated.

### First Embodiment

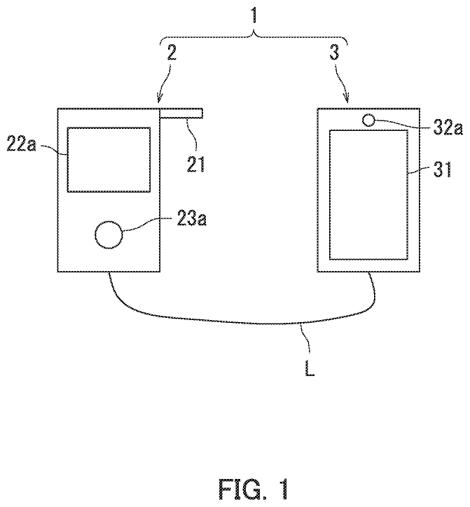

FIG. 1 is a schematic diagram illustrating a schematic configuration of an alcohol detection system 1 according to a first embodiment. As illustrated in FIG. 1, the alcohol detection system 1 includes an alcohol detector 2 and an information processing device 3 (external device). The information processing device 3 may be a mobile communication terminal such as a smartphone or a tablet terminal, or may be a personal computer. In the present embodiment, the following description takes a mobile communication terminal as an example of the information processing device 3.

The alcohol detector 2 and the information processing device 3 are communicatively connected via a communication cable L. A subject performs alcohol detection by himself or herself by operating the information processing device 3 and the alcohol detector 2, so that the alcohol detection system 1 performs the alcohol detection. The following describes the configurations of the alcohol detector 2 and the information processing device 3 in detail.

### (Alcohol Detector 2)

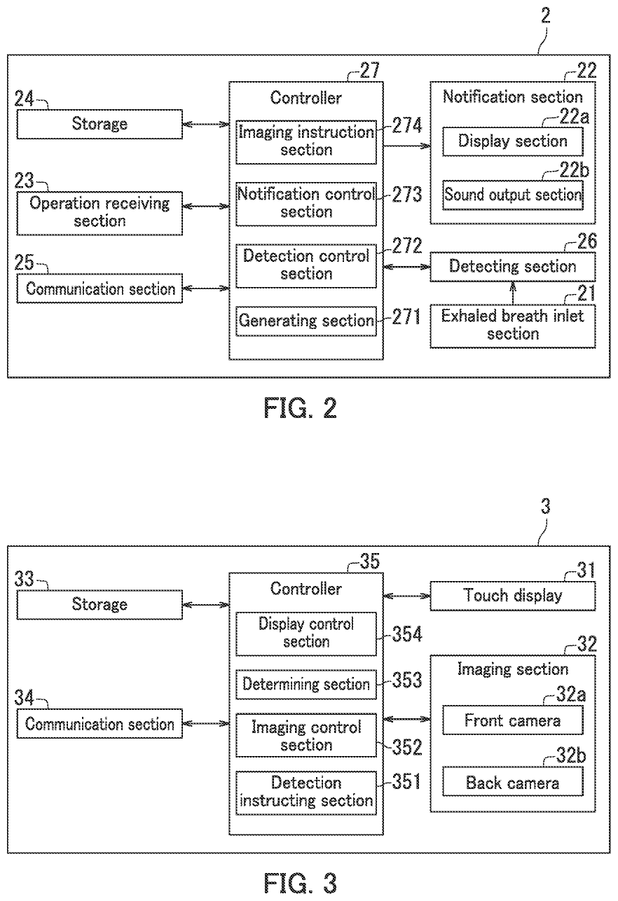

FIG. 2 is a block diagram illustrating a schematic configuration of the alcohol detector 2 illustrated in FIG. 1. The configuration of the alcohol detector 2 will be described with reference to FIGS. 1 and 2.

As illustrated in FIG. 2, the alcohol detector 2 includes an exhaled breath inlet section 21, a notification section 22, an operation receiving section 23, storage 24, a communication section 25, a detecting section 26, and a controller (first controller) 27.

The exhaled breath inlet section 21 is composed of a tubular member. As illustrated in FIG. 1, the exhaled breath inlet section 21 includes a portion protruding outward of the alcohol detector 2, and another portion connected to the detecting section 26 in the alcohol detector 2. A tip end of the portion protruding outward of the alcohol detector 2 is a mouthpiece. The subject puts the tip end of the portion protruding outward of the alcohol detector 2 in the mouth and blows air thereinto. The exhaled breath blown from the subject passes through the exhaled breath inlet section 21 to the detecting section 26.

The notification section 22 includes a display section 22a and a sound output section 22b. The display section 22a includes a liquid crystal display, an organic electroluminescent display, or the like, and displays an image under control of the controller 27. The sound output section 22b includes a speaker and outputs a sound such as a beep sound under control of the controller 27.

The operation receiving section 23 includes operation buttons such as a power button (not illustrated) of the alcohol detector 2 and a detection start button 23a (see FIG. 1). The operation receiving section 23 outputs, to the controller 27, an operation signal indicating an operation of pressing the operation button.

The storage 24 includes non-volatile memory such as electrically erasable programmable read-only memory (EEPROM (registered Japanese trademark)) or flash memory. The storage 24 stores therein various data necessary for the alcohol detector 2 to perform alcohol detection processing.

The communication section 25 includes a terminal (not illustrated) for connecting to the communication cable L (see FIG. 1). The communication section 25 communicates with the information processing device 3 via the communication cable L.

The detecting section 26 includes a pressure sensor and an alcohol sensor for alcohol concentration detection (both are not illustrated). In the present embodiment, a semiconductor type alcohol sensor is used as the alcohol sensor, for example.

However, the type of the sensor for alcohol concentration detection is not limited to the semiconductor type, and may be a fuel cell type, a non-dispersive infrared type, a chemical type, or the like.

The detecting section 26 further includes a heating mechanism (not illustrated) including a heater for heating the alcohol sensor. The detecting section 26 cleans the alcohol sensor by heating the alcohol sensor using the heating mechanism to remove residual gas and the like on the surface of the alcohol sensor before exhaled air is blown.

The controller 27 controls the detecting section 26 to detect the alcohol concentration in the exhaled air blown into the exhaled breath inlet section 21 at a pressure equal to or higher than a certain level.

The controller 27 includes a microcontroller (MCU) and memory (read-only memory (ROM) and random-access memory (RAM)) bus-connected to the MCU (both are not illustrated). The memory is an example of a non-transitory computer-readable storage medium. The MCU is connected to the notification section 22, the operation receiving section 23, the storage 24, the communication section 25, and the detecting section 26 via a bus.

Upon the execution of a control program stored in the ROM by the MCU, the controller 27 controls each section connected to the MCU for performing the alcohol detection processing. Specifically, upon execution of the control program by the MCU, the controller 27 functions as a generating section 271, a detection control section 272, a notification control section 273, and an imaging instruction section 274.

The generating section 271 generates a password as an example of identification information for identifying the alcohol detector 2. The generating section 271 randomly generates a password using at least one of alphabets, numbers, symbols, and the like based on a prescribed algorithm, and stores the password in the memory. Moreover, the generating section 271 transmits the password information indicating the generated password to the information processing device 3 via the communication section 25.

Once the detection start button 23a is pressed, the detection control section 272 causes the detecting section 26 to sequentially perform cleaning processing and alcohol detection processing. The detection control section 272 stores the detection result detected by the detecting section 26 in the memory, and transmits information indicating the detection result to the information processing device 3 via the communication section 25.

The notification control section 273 causes the notification section 22 to notify an image or a sound prompting pressing of the detection start button 23a in response to a detection start instruction from the information processing device 3 via the communication section 25. Additionally, the notification control section 273 causes the display section 22a to display an image (hereinafter referred to as detection result image) illustrating the detection result stored in the memory and an image (hereinafter referred to password image) indicating the password. The password image may represent the password by characters, or represent the password by a two-dimensional bar code or a QR code (registered Japanese trademark). Moreover, after the cleaning processing has completed, the notification control section 273 causes the notification section 22 to notify an image or a sound indicating completion of cleaning.

After acquiring the detection start instruction from the information processing device 3, the imaging instruction section 274 transmits a capture start signal instructing a start of image capture to the communication section 25 during the time between when the alcohol detection processing starts and when the alcohol detection processing ends.

### (Information Processing Device 3)

FIG. 3 is a block diagram illustrating a schematic configuration of the information processing device 3 illustrated in FIG. 1. The following describes the configuration of the information processing device 3 with reference to FIGS. 1 and 3.

As illustrated in FIG. 3, the information processing device 3 includes a touch display 31, an imaging section 32, a storage 33, a communication section 34, and a controller (second controller) 35. It should be noted that, in addition to the configuration illustrated in FIG. 3, the information processing device 3 also includes a microphone or a speaker for a call function, which is not illustrated in FIG. 3.

The touch display 31 is constituted by a display provided with a touch sensor. The display is constituted by a liquid crystal display, an organic electroluminescent display, or the like. The touch sensor is, for example, a capacitive touch sensor that detects a change in capacitance at a position where a user's finger or the like touches in a display area. The touch display 31 displays various images under control of the controller 35.

The imaging section 32 includes a front camera 32a and a back camera 32b. The front camera 32a and the back camera 32b each include a lens unit accommodating a plurality of lenses and an imaging element such as a charge coupled device (CCD) or a complementary metal oxide semiconductor (CMOS) image sensor. The front camera 32a captures an image of the subject from a first side of the information processing device 3 on which the touch display 31 is provided. The back camera 32b captures an image of the subject from a second side of the information processing device 3 opposite to the first side. The imaging section 32 has an imaging mode including a moving image mode for capturing a moving image and a still image mode for capturing a still image. Under control of the controller 35, the imaging section 32 performs image capture operation with the front camera 32a or the back camera 32b in a specified imaging mode.

The storage 33 includes non-volatile memory such as an embedded multi-media card (eMMC) or universal flash storage (UFS). Under control of the controller 35, the storage 33 stores various data including image data necessary for the alcohol detection and detection results of detection by the alcohol detector 2.

The communication section 34 includes a terminal (not illustrated) for connecting to the communication cable L. The communication section 34 communicates with the alcohol detector 2 via the communication cable L (see FIG. 1).

The controller 35 includes a central processing unit (CPU) and memory (ROM and RAM) connected to the CPU by a bus. The CPU is connected to the touch display 31, the imaging section 32, the storage 33, and the communication section 34 via a bus. Upon execution of the alcohol detection program stored in the ROM by the CPU, the controller 35 controls each section connected to the CPU for performing the alcohol detection processing. Specifically, upon execution of the alcohol detection program by the CPU, the controller 35 functions as a detection instructing section 351, an imaging control section 352, a determining section 353, and a display control section 354.

Once the alcohol detection program is activated by the subject, the detection instructing section 351 transmits a detection start signal indicating an alcohol detection start instruction to the alcohol detector 2 via the communication section 34.

The imaging control section 352 controls a start and a stop of the imaging section 32. In the present embodiment, the imaging control section 352 causes the imaging section 32 to start and stop based on imaging control information indicating a capture start or a capture end from the alcohol detector 2. Specifically, when having acquired, as the imaging control information, a capture start signal from the alcohol detector 2, the imaging control section 352 activates the front camera 32a in the moving image mode, and stores image data captured by the front camera 32a in the memory. Moreover, the imaging control section 352 causes the front camera 32a to end its operation based on imaging control information indicating an imaging end. In the present embodiment, the imaging control information indicating an imaging end is a detection result transmitted from the alcohol detector 2.

The determining section 353 acquires the detection result from the alcohol detector 2 via the communication section 34 and stores it in the memory. Moreover, the determining section 353 acquires the password information from the alcohol detector 2 via the communication section 34 and stores it in the memory. The determining section 353 compares the password information with the image data stored in the memory, and determines validity of the alcohol detector 2 for alcohol detection. Specifically, the determining section 353 determines that the alcohol detector 2 is invalid when the image data does not include an image matching the password indicated by the password information. Moreover, when the image data includes an image matching the password, the determining section 353 compares the image data with a predetermined face image of the subject to determine validity of the subject. Specifically, the determining section 353 determines that the subject is invalid when the image data does not include an image matching the face image of the subject. When at least one of the alcohol detector 2 and the subject is invalid, the determining section 353 determines that the acquired detection result is invalid, and invalidates the detection result.

The display control section 354 causes the touch display 31 to display an alcohol detection screen indicating a start of alcohol detection in response to activation of the alcohol detection program by the subject. The display control section 354 causes the touch display 31 to display an image being captured by the imaging unit 32. The display control section 354 also causes the touch display 31 to display a result of determination by the determining section 353.

### Operation

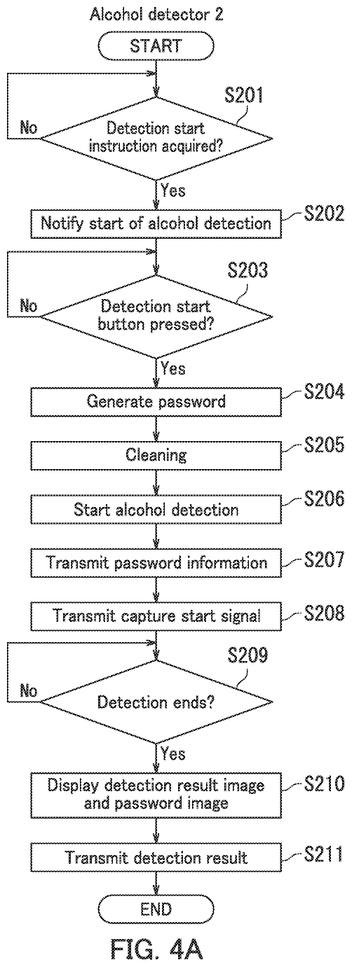

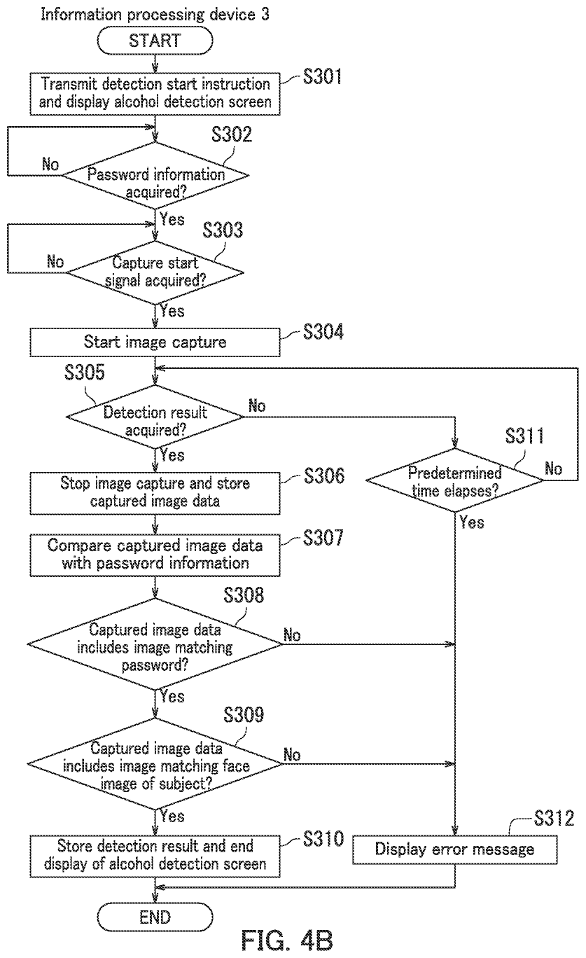

The following describes the operation of the alcohol detection processing of the alcohol detection system 1. FIG. 4A is a flowchart illustrating an operation of the alcohol detector 2. FIG. 4B is a flowchart illustrating an operation of the information processing device 3. The operation of the information processing device 3 according to the flow illustrated in FIG. 4B starts in response to activation of the alcohol detection program by the subject. The following describes the alcohol detection processing with reference to FIGS. 1 to 4B.

In Step S301 of FIG. 4B, in response to the activation of the alcohol detection program, the information processing device 3 transmits a signal indicating a detection start instruction to the alcohol detector 2, and displays the alcohol detection screen on the touch display 31. Specifically, the detection instructing section 351 of the information processing device 3 transmits a signal indicating the detection start instruction to the alcohol detector 2 via the communication section 34. Thereafter, the display control section 354 causes the touch display 31 to display the alcohol detection screen for notifying of the subject an alcohol detection start.

In Step S201 of FIG. 4A, upon receiving the detection start signal from the information processing device 3 via the communication section 25 (Step S201: Yes), the alcohol detector 2 notifies the alcohol detection start (Step S202). Specifically, the notification control section 273 of the alcohol detector 2 causes the display section 22a to display an image prompting pressing of the detection start button 23a (see FIG. 1), or causes the sound output section 22b to output a sound prompting pressing of the detection start button 23a.

The alcohol detector 2 is in standby until the subject presses the test start button 23a (Step S203: No). Once the test start button 23a is pressed by the subject (Step S203: Yes), the alcohol detector 2 generates a password (Step S204). Specifically, the generating section 271 of the alcohol detector 2 acquires an operation signal indicating pressing of the detection start button 23a from the operation receiving section 23, generates a password based on the prescribed algorithm, and stores it in the memory.

Subsequently, the alcohol detector 2 performs cleaning processing (Step S205). Specifically, the detection control section 272 of the alcohol detector 2 heats the heating mechanism (not illustrated) in the detecting section 26 to clean the alcohol sensor.

After the cleaning processing, the alcohol detector 2 causes the display section 22a to display an image indicating an end of the cleaning processing, and causes the detecting section 26 to start the alcohol detection processing (Step S206). The generating section 271 of the alcohol detector 2 transmits the password information indicating the password stored in the memory to the information processing device 3 via the communication section 25 (Step S207). Then, the imaging instruction section 274 of the alcohol detector 2 transmits the capture start signal to the information processing device 3 via the communication section 25 (Step S208). It should be noted that the password information and the capture start signal may be transmitted substantially simultaneously.

The detecting section 26 of the alcohol detector 2 keeps performing alcohol concentration detection until the alcohol concentration in the exhaled breath blown from the exhaled breath inlet section 21 at a certain pressure or higher is detected (Step S209: No).

In Step S302 of FIG. 4B, upon acquiring the password information from the alcohol detector 2 via the communication section 34 (Step S302: Yes) and acquiring a capture start signal (Step S303: Yes), the information processing device 3 stores the acquired password information in the memory, and the imaging control section 352 causes the imaging section 32 to start image capture (Step S304). Specifically, the imaging control section 352 of the information processing device 3 causes the front camera 32a to start image capture in the moving image mode. The display control section 354 causes the touch display 31 to display the image captured by the front camera 32a.

That is, the information processing device 3 starts performing image capture in a time between when the alcohol detector 2 starts the alcohol detection processing and the time when the alcohol detector 2 ends the alcohol detection processing. At that time, the subject holds the information processing device 3 in one hand and confirms the image displayed on the touch display 31 to make sure that the subject himself or herself and the alcohol detector 2 can be captured by the front camera 32a, while the other hand holds the alcohol detector 2 to exhale breath into the exhaled breath inlet section 21 of the alcohol detector 2.

It should be noted that in Step S302, the information processing device 3 is in standby with the alcohol detection screen displayed until the password information is acquired (Step S302: No). In Step S303, the information processing device 3 is in standby until the capture start signal is acquired (Step S303: No).

Returning to FIG. 4A, if the detecting section 26 has completed the alcohol detection processing (Step S209: Yes), the alcohol detector 2 causes the display section 22a to display the detection result image and the password image (Step S210). In detail, the notification control section 273 of the alcohol detector 2 causes the display section 22a to display the detection result image indicating the detection result detected by the detecting section 26 and the password image indicating the password stored in the memory. The password image is a QR code (registered Japanese trademark) generated using a prescribed encoder, for example. Thereafter, the detection control section 272 of the alcohol detector 2 transmits the detection result via the communication section 25.

In Step S305 of FIG. 4B, if the information processing device 3 has acquired the detection result from the alcohol detector 2 via the communication section 34 (Step S305: Yes), the imaging control section 352 causes the imaging section 32 to stop image capture, and stores captured image data obtained by image capture in the storage 33 (Step S306).

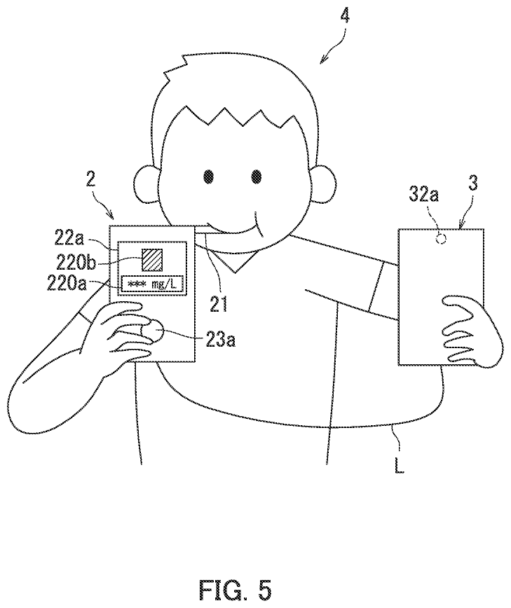

FIG. 5 is a schematic diagram illustrating a situation in which the subject is performing alcohol detection. More specifically. FIG. 5 illustrates that the subject 4 is shooting an image using the front camera 32a of the information processing device 3 while the password image 220b and the detection result image 220a indicating the detection result, which is detected as a result of the subject blowing breath into the alcohol detector 2 are displayed on the display section 22a. As described above, in the present embodiment, an image of the alcohol detector 2 and the subject 4 is captured not only during alcohol concentration detection but also during the period when the detection result image 220a and the password image 220b are displayed after the detection. In detail, the information processing device 3 continues image capture during the time when the detection result image 220a and the password image 220b are displayed on the display section 22a. The information processing device 3 stops image capture when the alcohol detector 2 acquires the detection result.

Returning to FIG. 4B, the determining section 353 of the information processing device 3 compares the captured image data stored in the storage 33 with the password information, that is, the password stored in the memory (Step S307).

The determining section 353 determines whether or not the captured image data includes an image that matches the password (Step S308). Specifically, for example, when the captured image data contains the password image 220b represented by a QR code (registered Japanese trademark), the determining section 353 decodes the QR code (registered Japanese trademark) contained in the captured image data using a prescribed analysis algorithm. The determining section 353 determines whether or not the decoded result matches the password.

When the determining section 353 of the information processing device 3 determines that the decoded results does not match the password and the captured image data does not contain an image matching the password (Step S308: No), the display control section 354 causes the touch display 31 to display an error message (Step S312). In other words, when the captured image data does not contain the image matching the password, it is determined that the alcohol detector 2 contained in the captured image data is invalid, and the acquired detection result will not be stored in the storage 33. The error message indicates that the alcohol detection has been completed abnormally, for example.

In Step S308, upon determining that the decoded result matches the password and the captured image data contains the image matching the password (Step S308: Yes), the determining section 353 determines whether or not the captured image data contains an image matching the predetermined face image of the subject (Step S309). Upon determining that the captured image data contains the image matching the face image of the subject (Step S309: Yes), the determining section 353 of the information processing device 3 causes the storage 33 to store the detection result stored in the memory. Thereafter, the information processing device 3 ends display of the alcohol detection screen by the display control section 354, and notifies the subject of an alcohol detection end (Step S310).

In Step S309, when the determining section 353 has determined that the captured image data does not contain an image matching the face image of the subject (Step S309: No), the processing of the information processing device 3 proceeds to Step S312.

In Step S305, the information processing device 3 is in standby until the detection result is acquired from the alcohol detector 2 via the communication section 34 within a predetermined time (Step S305: No. Step S311: No). If the information processing device 3 has not acquired the detection result from the alcohol detector 2 via the communication section 34 within the predetermined time (Step S305: No, Step S311: Yes), the information processing device 3 performs the process in Step S312 after the predetermined time has elapsed. It should be noted that the predetermined time is about ten and several seconds, for example.

The operation of the alcohol detection processing in the alcohol detection system 1 of the present embodiment has been described. For example, a case of alcohol detection deception may be assumed that a true subject pretending to perform alcohol detection using another person's alcohol detector shoots an image of himself or herself using the information processing device 3 simultaneously with the other person pretending to be the true subject blows breath into the true subject's alcohol detector 2. In this case, the captured image data captured by the information processing device 3 does not contain an image matching the password generated by the true subject's alcohol detector 2. Therefore, the information processing device 3 determines that the detection result transmitted from the subject's alcohol detector 2 to the image processing device 3 is invalid, and causes the touch display 31 to display an error message. In other words, the information processing device 3 determines identity fraud of the alcohol detector 2, and invalidates the detection result. Therefore, according to the alcohol detection system 1, the alcohol detector 2 is difficult to use for alcohol detection deception, and validity determination of the detection result can be more reliable compared with the case in which validity determination of the detection result based on only the face authentication of the subject. In other words, identity fraud of a alcohol detector can be prevented.

As a reference example, deception of the subject can be prevented to a certain extent by capturing an image of the face of the subject who is performing the alcohol detection, and determining the validity of the subject based on the captured image. However, there is a possibility that the subject may capture an image of another person's alcohol detector for detecting the alcohol concentration replaced with the subject's alcohol detector, and the other person exhales breath into the subject's alcohol detector. According to the reference example, it is difficult to reliably determine whether or not the detection result is valid because the subject in the captured image is valid due to identity fraud of the alcohol detector. However, in the present embodiment, as described above, alcohol detection by identity fraud of the alcohol detector can be prevented.

Moreover, the alcohol detector 2 of the present embodiment generates a different password each time alcohol detection is performed. Therefore, it can be ensured that the identity fraud of the alcohol detector is prevented compared with the case using the same password for each alcohol detection.

### Second Embodiment

A second embodiment describes transmission timing of the password information and the capture start signal that are different from the first embodiment.

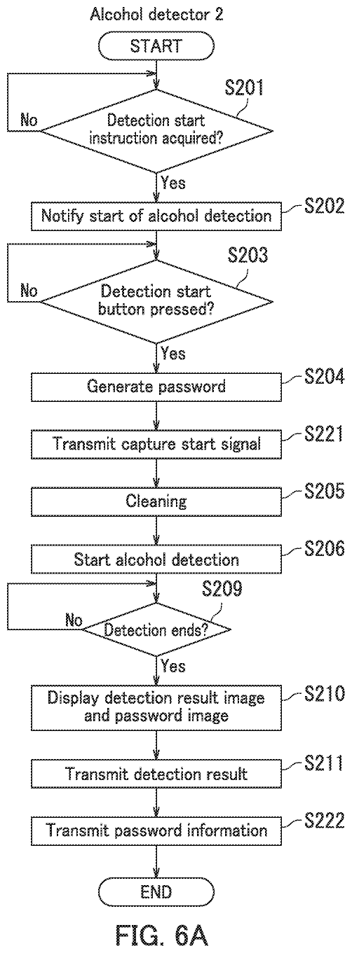

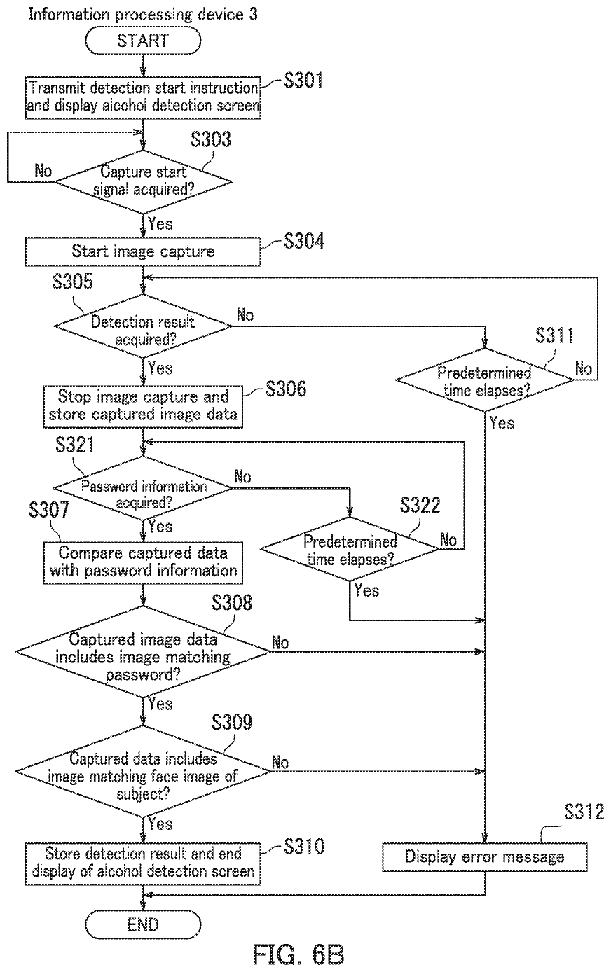

FIG. 6A is a flowchart illustrating an operation example of the alcohol detector 2 in the present embodiment. FIG. 6B is a flowchart illustrating an operation example of the information processing device 3 in the present embodiment. In FIGS. 6A and 6B, the same steps as those in the first embodiment are designated by the same reference signs as those in the first embodiment. The following mainly describes the steps different from those of the first embodiment.

In FIG. 6A, the alcohol detector 2 performs the processing of Steps S201 to S204. After the generating section 271 stores a password generated in Step S204 in the memory, the alcohol detector 2 causes the imaging instruction section 274 to transmit a capture start signal to the information processing device 3 via the communication section 25 (Step S221).

In FIG. 6B, after Step S301 is completed, the information processing device 3 acquires the capture start signal from the alcohol detector 2 via the communication section 34 (Step S303: Yes), and the imaging control section 352 causes the imaging section 32 to start image capture (Step S304).

Returning to FIG. 6A, after transmitting the capture start signal (Step S221), the alcohol detector 2 performs Steps S205, S206, and S209 to S211. After the alcohol detector 2 has transmitted the detection result to the information processing device 3, the generating section 271 transmits password information indicating a password stored in the memory to the information processing device 3 via the communication section 25 (Step S222).

In FIG. 6B, upon acquiring the detection result from the alcohol detector 2 via the communication section 34 (Step S305: Yes), the processing of the information processing device 3 proceeds to Step S306. Thereafter, upon acquiring the password information from the alcohol detector 2 via the communication section 34 (Step S321: Yes), the information processing device 3 performs Steps S307 to S310. Moreover, the information processing device 3 is in standby for a predetermined period of time if the information processing device 3 does not acquire the password information from the alcohol detector 2 via the communication section 34 until the predetermined period of time has elapsed (Step S321: No. Step S322: No), and the process proceeds to Step S312 after the predetermined period of time elapses (Step S322: Yes).

That is, in the present embodiment, the capture start signal is transmitted to the information processing device 3 before the alcohol detection starts, and the information processing device 3 starts image capture before the alcohol detection starts. In the present embodiment, the alcohol detector 2 transmits the password information after transmitting the detection result to the information processing device 3, that is, after the alcohol detection is completed. Even in this configuration, the information processing device 3 performs image capture during the detection result and the password image being displayed in the alcohol detector 2 as in the first embodiment. Therefore, identity fraud of the alcohol detector 2 and impersonation of the subject can be prevented.

### Third Embodiment

A third embodiment describes an example in which the timing of password generation in the alcohol detector 2 is different from that in the second embodiment.

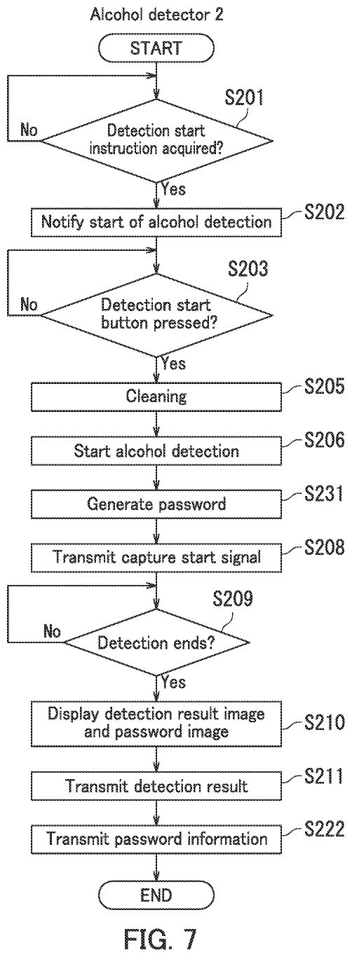

FIG. 7 is a flowchart illustrating an operation example of the alcohol detector 2 in the present embodiment. In FIG. 7, the same steps as those of the first and second embodiments are designated by the same reference signs as those of the first and second embodiments. In the present embodiment, since the operation of the information processing device 3 is the same as that of the second embodiment (FIG. 6B), the description thereof will be omitted.

The alcohol detector 2 performs Steps S201 to 203, S205, and S206. Then, the alcohol detector 2 causes the generating section 271 to generate a password, and stores the password in the memory (Step S231). Thereafter, the alcohol detector 2 causes the imaging instruction section 274 to transmit a capture start signal to the information processing device 3 via the communication section 25 (Step S208).

The detecting section 26 performs alcohol concentration detection until the alcohol concentration has been detected (Step S209: No). After the alcohol detection has been completed (Step S209: Yes), the alcohol detector 2 performs Steps S210 and S211. Thereafter, the generating section 271 of the alcohol detector 2 transmits, to the information processing device 3 via the communication section 25, password information indicating the password stored in the memory (Step S222).

As described above, in the present embodiment, the alcohol detector 2 generates a password during alcohol detection, and transmits the password information to the information processing device 3 after transmitting the detection result to the information processing device 3. It should be noted that the timing for password generation is not limited to this. For example, after alcohol detection is completed, the alcohol detector 2 may generate a password before or after transmission of the detection result.

### Fourth Embodiment

A fourth embodiment describes a case where the information processing device generates a password.

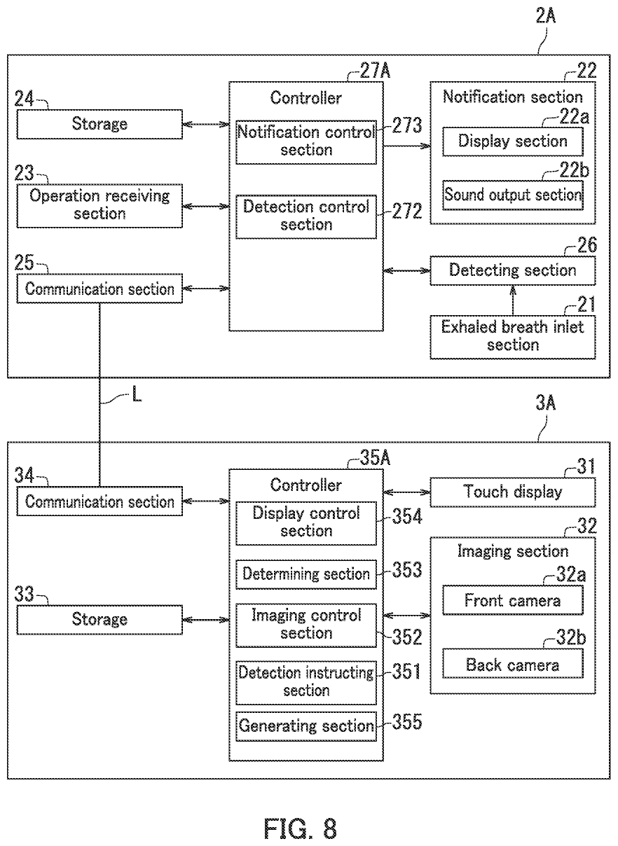

FIG. 8 is a block diagram illustrating a schematic configuration of an alcohol detector 2A and an information processing device 3A in the present embodiment. In FIG. 8, the same configurations as those of the first embodiment are designated by the same reference signs as those of the first embodiment. The following mainly describes a configuration different from that of the first embodiment.

As illustrated in FIG. 8, the alcohol detector 2A includes a controller 27A. The controller 27A includes a detection control section 272 and a notification control section 273 similar to those of the first embodiment.

The information processing device 3A includes a controller 35A. The controller 35A includes a generating section 355 in addition to a detection instructing section 351, an imaging control section 352, a determining section 353, and a display control section 354 similar to those of the first embodiment.

The generating section 355 generates a password as identification information for identifying the alcohol detector 2A based on a prescribed algorithm, and transmits password information containing the generated password to the alcohol detector 2A via the communication section 34. The password is randomly generated each time alcohol detection is performed, similarly to that of the first embodiment.

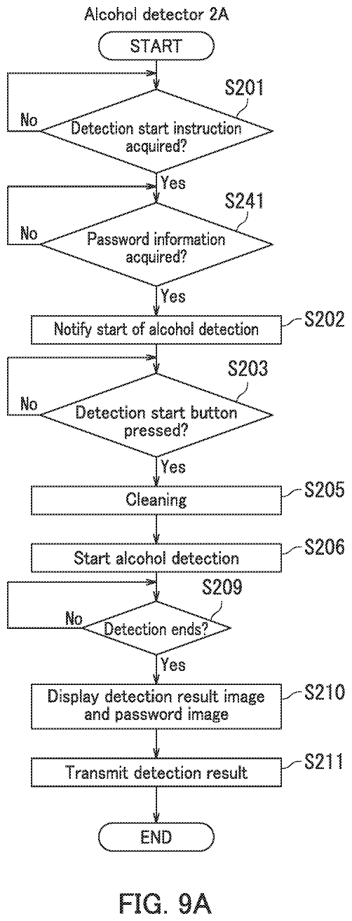

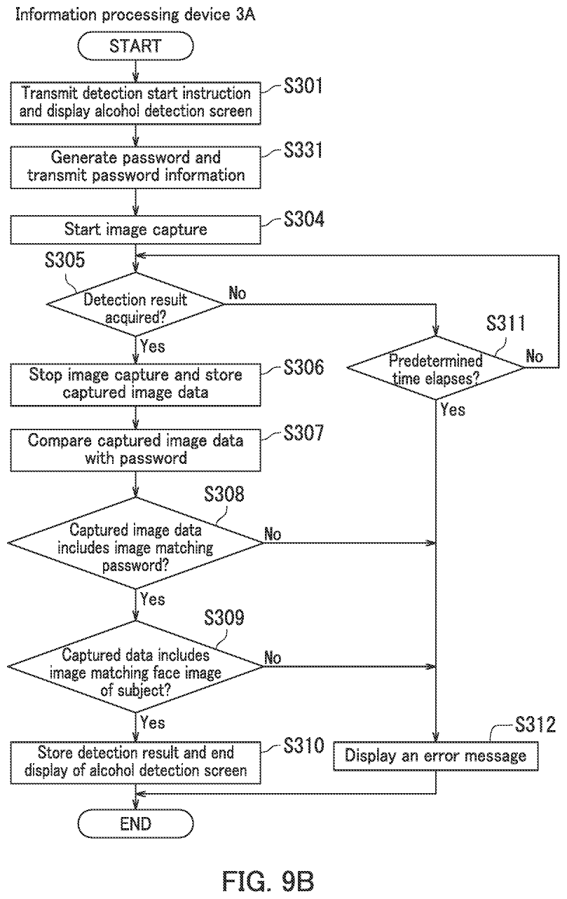

FIG. 9A is a flowchart illustrating an operation example of the alcohol detector 2A of the present embodiment. FIG. 9B is a flowchart illustrating an operation example of the information processing device 3A of the present embodiment. In FIGS. 9A and 9B, the same steps as those of the first embodiment are designated by the same reference signs as those of the first embodiment.

In FIG. 9B, after the process of Step S301, the generating section 355 of the information processing device 3A generates a password, stores it in the memory, and transmits password information containing the password to the alcohol detector 2A via the communication section 34 (Step S331). Thereafter, the imaging control section 352 of the information processing device 3A causes the imaging section 32 to start image capture (Step S304).

In FIG. 9A, if the alcohol detector 2A acquires, from the information processing device 3A via the communication section 25, a signal indicating a detection start instruction (Step S201: Yes) and the password information (Step S241: Yes), the alcohol detector 2A performs Steps S202 and the steps thereafter (Steps S203, S205, S206, and S209). That is, in the present embodiment, the alcohol detector 2A performs alcohol detection based on the acquisition of the password information from the information processing device 3A. Moreover, in Step S241, the alcohol detector 2A is in standby until the password information is acquired (Step S241: No).

In FIG. 9B, the information processing device 3A starts image capture (Step S304), and then performs the processes of Steps S305 to S307 and S311 likewise in the first embodiment. In other words, the information processing device 3A stops image capture upon acquiring the detection result from the alcohol detector 2A. Thereafter, the information processing device 3A performs Steps S308 to S310 and S312 likewise in the first embodiment.

### Fifth Embodiment

The identification information for alcohol detector identification is not limited to the password that is automatically generated using a prescribed algorithm. The image capture control information for instructing the information processing device to start image capture is not limited to the capture start signal. In a fifth embodiment, other examples of the identification information and the image capture control information indicating the start of image capture will be described.

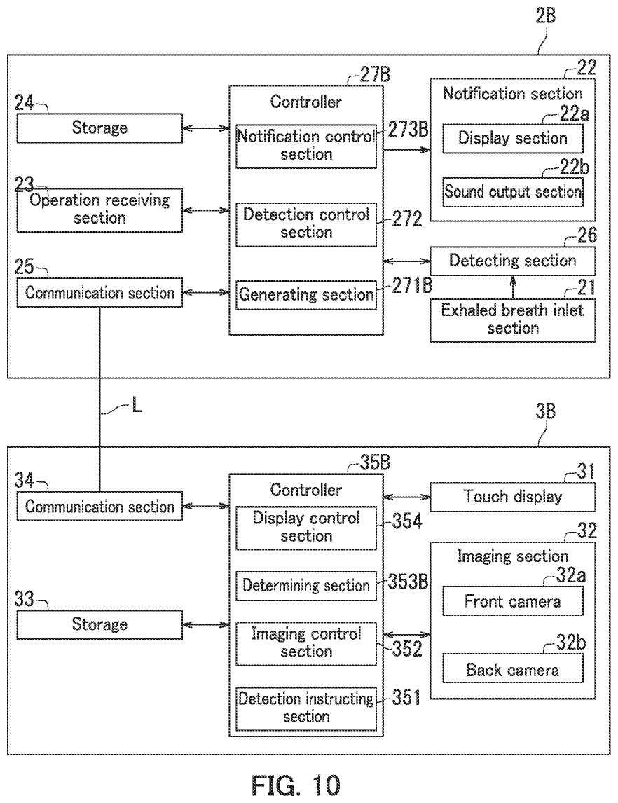

FIG. 10 is a block diagram illustrating a configuration example of an alcohol detector 2B and an information processing device 3B in the present embodiment. In FIG. 10, the same configurations as those of the first embodiment are designated by the same reference signs as those of the first embodiment.

As illustrated in FIG. 10, the alcohol detector 2B includes a controller 27B. The controller 27B includes a notification control section 273B and a generating section 271B in addition to a detection control section 272 similar to that of the first embodiment.

Where the detection start button 23a is pressed, the generating section 271B converts a system time measured by the alcohol detector 2B into a calendar time in nanosecond unit. Thereafter, the generating section 271B stores, as the identification information of the alcohol detector 2, internal time information containing the nanosecond portion of the calendar time in the memory.

After the detecting section 26 detects the alcohol concentration, the notification control section 273B causes the display section 22a to display the detection result and an internal time image indicating the internal time information.

As illustrated in FIG. 10, the information processing device 3B includes a controller 35B. The controller 35B includes a determining section 353B in addition to a detection instructing section 351, a imaging control section 352, and a display control section 354 similar to those of the first embodiment.

The determining section 353B compares the internal time information transmitted from the alcohol detector 2B with the captured image data of the image captured by the imaging section 32. When the captured image data contains an image matching the internal time information, the determining section 353B determines that the detection result detected by the alcohol detector 2B is valid.

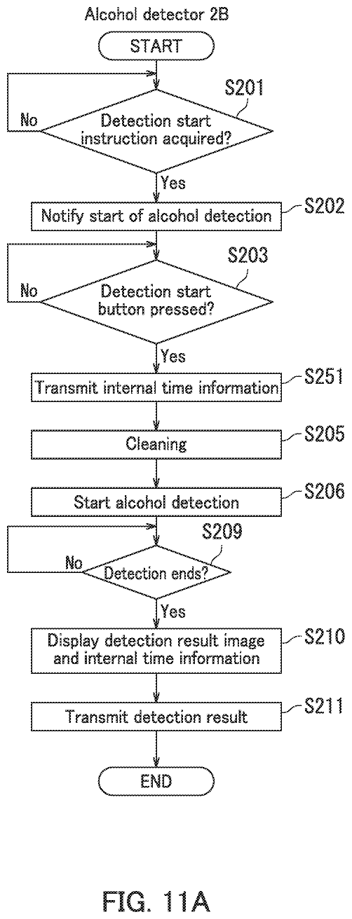

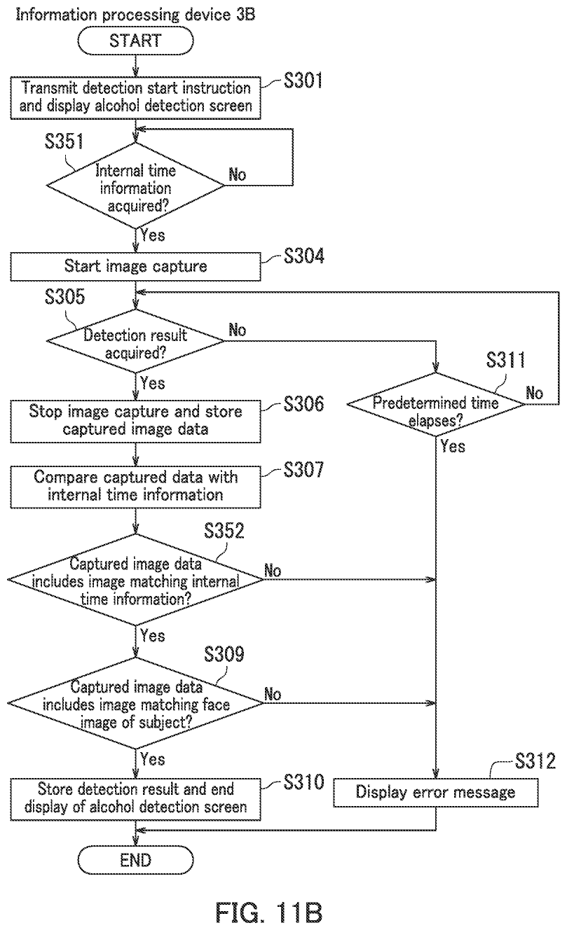

FIG. 11A is a flowchart illustrating an operation example of the alcohol detector 2B of the present embodiment. FIG. 11B is a flowchart illustrating an operation example of information processing device 3B of the present embodiment. In FIGS. 11A and 11B, the same steps as those in the first embodiment are designated by the same reference signs as those of the first embodiment.

In FIG. 11B, the information processing device 3B performs Step S301 and then is in standby until the internal time information is acquired from the alcohol detector 2B (Step S351: No).

In FIG. 11A, the alcohol detector 2B performs Steps S201 and 202, and if receives pressing of the detection start button 23a (Step S203: Yes), the generating section 271B generates the internal time information and transmits it via the communication section 25 (Step S241).

In FIG. 11B, if the information processing device 3B acquires the internal time information from the alcohol detector 2B via the communication section 34 (Step S351: Yes), the imaging control section 352 causes the imaging section 32 to start image capture (Step S304). That is, in the present embodiment, the internal time information is used as the image capture control information indicating the start of image capture, and the information processing device 3B starts image capture in response to acquisition of the internal time information.

Returning to FIG. 11A, after transmitting the internal time information in Step S241, the alcohol detector 2B performs Steps S205, S206, and S209. After the alcohol detector 2B completes detection, the notification control section 273B causes the display section 22a to display the detection result and an internal time image indicating the internal time information (Step S210).

In FIG. 11B, upon acquiring the detection result from the alcohol detector 2B (Step S305: Yes), the information processing device 3B performs Steps S306 and S307. Thereafter, the determining section 353B determines whether or not the captured image data obtained by the imaging section 32 contains an image matching the internal time information stored in the memory (Step S352), and the processing proceeds to Step S309 or Step S312 according to the determination result.

In this embodiment, the internal time information indicating the nanosecond portion of the calendar time specified from the system time in the alcohol detector 2B after pressing of the detection start button 23a is used as the identification information of the alcohol detector 2B. In this way, the identification information using the nanosecond portion of the calendar time is difficult to be inferred, thereby preventing identity fraud and impersonation. The information processing device 3B starts image capture in response to acquisition of the internal time information from the alcohol detector 2B, and stops image capture in response to acquisition of the detection result from the alcohol detector 2B. The image capture operation is performed during alcohol detection and during the detection result and the internal time image being displayed. Therefore, identity fraud of the alcohol detector and impersonation of the subject are difficult to be done. It should be noted that the internal time information is not limited to indicating a nanosecond portion of the calendar time of the alcohol detector 2B, and may indicate a microsecond portion of the calendar time, for example. The internal time information is preferably an internal time value that is difficult for a third person to infer.

The embodiments of the alcohol detection system, the alcohol detector, the information processing device, and the program have been described above with reference to the drawings (FIGS. 1 to 11B). However, the alcohol detection system, the alcohol detector, the information processing device, and the program are not limited to the above embodiments, and can be implemented in various embodiments without departing from the gist thereof. The drawings mainly illustrate various constituent elements schematically for ease of understanding. Aspects such as thickness, length, and number of each constituent element illustrated in the drawings may differ in practice for convenience of drawing preparation. Furthermore, aspects such as material, shape, and dimension of each constituent element illustrated in the above embodiments are merely examples and not particular limitations. Various changes can be made without substantially deviating from the effects of the present disclosure. The following describes variations of the above-described embodiments.

### Variations

(1) The alcohol detectors 2, 2A and 2B and the respective information processing devices 3, 3A and 3B are connected through wired connection by the communication cable L (see FIG. 1), but may be connected through wireless connection by Bluetooth (registered Japanese trademark), a wireless LAN, or the like.

(2) In the first to fifth embodiments, the imaging mode for image capture by the information processing devices 3, 3A, and 3B is not limited to the moving image mode, but may be the still image mode. In other words, the information processing devices 3, 3A, and 3B may perform image capture in the still image mode during the password information being displayed on the display section 22a of the alcohol detector 2.

(3) In the first to fifth embodiments, the display section 22a of the alcohol detector 2 displays the detection result image and the password image. However, the display section 22a may display at least one of the password image and the internal time image.

(4) In the first to fifth embodiments, it is possible that the information processing devices 3, 3A and 3B capture an image of the face of the subject at an alcohol detection start and authenticate the subject based on the captured image data obtained by image capture and the predetermined face image of the subject. Specifically, in Step S301, the information processing devices 3, 3A, and 3B cause the touch display 31 to display capture of the face of the subject while displaying the alcohol detection screen, and causes the imaging section 32 to perform image capture in the still image mode or the moving image mode. The information processing devices 3, 3A, and 3B may determine that the subject is invalid when the captured image data does not contain an image matching the predetermined face image of the subject and cause the touch display 31 to display an error message. According to the above configuration, subject impersonation can be prevented by capturing an image of the face of the subject in alcohol detection and determining validity of the subject.

(5) The information processing devices 3, 3A, and 3B may be connected to a server managed by an operation company providing services of for example taxis or buses via a communication network such as the Internet. In this case, the information processing devices 3, 3A and 3B may transmit, to the server, the information indicating the detection result acquired from the alcohol detectors 2, 2A and 2B together with the identification information of the subject. According to the above configuration, the operation company can manage the alcohol detection results of drivers working for the operation company for each of the drivers.

(6) The information processing devices 3, 3A and 3B may include, for example, a vibrator (not illustrated) as the notification section in addition to the touch display 31 and the speaker (not illustrated). In Step S312 (FIGS. 4B, 6B, 9B, and 11B), the information processing devices 3, 3A, and 3B may cause the touch display 31 to display an error message, or may cause the speaker to output a notification sound indicating an error. Alternatively, the information processing devices 3, 3A, and 3B may cause the vibrator to vibrate at a set frequency indicating an error. In other words, the information processing device 3 may notify of the error by using at least one of an image, a sound, and vibration.

(7) In the first to fifth embodiments, the information processing devices 3, 3A and 3B store the alcohol detection program. However, the alcohol detection program may be recorded on a removable media and the information processing devices 3, 3A and 3B may read out the alcohol detection program from the removable media. Alternatively, the alcohol detection program may be stored in an external server or the like and the information processing devices 3, 3A, and 3B may download the alcohol detection program via a communication network such as the Internet.

(8) In the first to fifth embodiments, the alcohol detector may have the same function as the determining section 353 of the information processing devices 3, 3A, and 3B to determine the validity of each of the captured image of the subject under alcohol detection and the detection result. In this case, the alcohol detector performs alcohol detection in response to a detection operation start by the subject. The alcohol detector causes the display section 22 to display the identification information from a start to an end of alcohol detection. The alcohol detector may acquire an image of the subject captured by a camera connected to the alcohol detector through wired connection or wireless connection during the identification information being displayed. Alternatively, the alcohol detector may acquire an image of the subject captured by an information processing device (smartphone or the like) communicatively connected to the alcohol detector. The alcohol detector determines validity of the detection result based on the detection result, the identification information, and the captured image.

(9) In the fifth embodiment, the information processing device 3B may generate the internal time information based on the system time of the information processing device 3B, and transmit it to the alcohol detector 2B. In this case, the alcohol detector 2B and the information processing device 3B may perform operation according to the operation flow of the fourth embodiment (FIGS. 9A and 9B) in which the password information and the password image are replaced with the internal time information and the internal time image. In the first to third embodiments, the internal time information of the alcohol detector may be used instead of the password.

The identification information may be any information as long as it can uniquely identify the alcohol detector by the information processing device. In other words, the information processing device and the alcohol detector may store in advance common information as identification information for identifying the alcohol detector. From the viewpoint of security, the identification information stored in both the information processing device and the alcohol detector is preferably changed each time alcohol detection is performed.

(10) If No in Step S302 or Step S303 of FIGS. 4B and 6B, and if No in Step S351 of FIG. 11B, the information processing devices 3 and 3B may be in standby for a predetermined time, and performs Step S312 after the predetermined time elapses. Moreover, if No in Step S241 of FIG. 9A, Step S203 of FIGS. 4A, 6A, 7, and 11A, and No in Step S209, the alcohol detectors 2, 2A and 2B may be in standby for a predetermined time, and end alcohol detection after the predetermined time elapses.

(11) In FIGS. 4B, 6B, 9B, and 11B, the information processing devices 3, 3A and 3B may repeat determination of Step S305 at regular time intervals (e.g., 1-second intervals). If a negative determination is made until determination of Step S305 is repeated by a predetermined number of times (e.g., 10 times), the processing of the information processing device 3, 3A or 3B may proceed to Step S312. Similarly, in determination in each step in the above variation (10), determination is repeated until the number of times of repetition reaches a predetermined times. If a negative determination is made until the number of times of repetition reaches the predetermined times, the processing of the information processing device 3, 3A or 3B may proceed to Step S312 or ends alcohol detection.

(12) In Step S301 (FIG. 4B, FIG. 6B, FIG. 9B, and FIG. 11B), the information processing devices 3, 3A, and 3B may display a message prompting pressing of the detection start button 23a of the alcohol detector 2 while causing display of the alcohol detection screen. In this case, the information processing devices 3, 3A, and 3B may not transmit the detection start instruction. Moreover, in this case, the alcohol detectors 2, 2A and 2B may not perform Steps S201 and S202 (FIGS. 4A, 6A, 7, 9A, 11A).

What is claimed is:

1. An alcohol detector comprising:
a detecting section configured to perform alcohol concentration detection, the alcohol concentration detection detecting alcohol concentration in exhaled breath blown by a subject;
a display section;
a communication section configured to communicate with a prescribed external device performing image capture of the subject who blows the exhaled breath; and
a controller configured to cause the detecting section to perform the alcohol concentration detection and transmit a detection result of the alcohol concentration detection to the prescribed external device via the communication section, wherein,
the controller causes the display section to display an image including identification information indicating the alcohol detector during the image capture of the subject in a time from a start of the alcohol concentration detection to transmission of the detection result.

2. The alcohol detector according to claim 1, wherein,
the controller generates a different identification information piece as the identification information each time the alcohol concentration detection is performed, and causes the display section to display an image indicating the generated identification information piece.

3. The alcohol detector according to claim 1, wherein,
the identification information contains internal time information based on a system time of the alcohol detector.

4. The alcohol detector according to claim 1, wherein,
the controller transmits the identification information to the prescribed external device via the communication section before or after the image indicating the identification information is displayed by the display section.

5. The alcohol detector according to claim 1, wherein,
the controller acquires the identification information from the prescribed external device.

6. The alcohol detector according to claim 1, wherein,
the controller causes the display section to display an image indicating the detection result.

7. An information processing device comprising:
a communication section configured to communicate with an alcohol detector that performs alcohol concentration detection of exhaled breath of a subject, displays an image including identification information indicating the alcohol detector, and outputs a detection result of the alcohol concentration detection;
an imaging section; and
a controller, wherein,
the controller:
causes the imaging section to perform image capture during display of an image including at least the identification information before the detection result is output by the alcohol detector;
acquires the detection result of the alcohol concentration detection via the communication section after the imaging section starts the image capture; and
determines whether or not the acquired detection result is valid based on the identification information and a captured image captured by the imaging section, and
in determining whether or not the detection result is valid, the controller determines that the detection result is invalid when the captured image does not include an image matching the identification information.

8. The information processing device according to claim 7, wherein,
the controller acquires image capture start information indicating a start of image capture from the alcohol detector, and causes the imaging section to start image capture based on the acquired image capture start information.

9. The information processing device according to claim 7, wherein,
in determining whether or not the detection result is valid, the controller determines that the detection result is invalid when the captured image does not include an image matching a predetermined image of the subject.

10. The information processing device according to claim 7, wherein,
the captured image is a moving image.

11. The information processing device according to claim 7, wherein,
the captured image is a still image.

12. The information processing device according to claim 7, further comprising
a notification section configured to perform notification based on an input signal indicating an error using at least one of a sound, an image, and vibration, wherein
when determining that the detection result is invalid, the controller inputs the input signal to the notification section.

13. A non-temporary computer-readable storage medium that stores therein a program causing an information processing device to execute processing, the information processing device including an imaging section and a communication section communicating with an alcohol detector that performs alcohol concentration detection of exhaled breath of a subject, displays an image including identification information indicating the alcohol detector, and outputs a detection result of the alcohol concentration detection, wherein,
the processing includes:
causing the imaging section to perform image capture during display of an image including at least the identification information before the detection result is output by the alcohol detector;
acquiring the detection result of the alcohol concentration detection via the communication section after the imaging section starts the image capture; and
determining whether or not the acquired detection result is valid based on the identification information and a captured image captured by the imaging section, and
in determining whether or not the detection result is valid, it is determined that the detection result is invalid when the captured image does not include an image matching the identification information.

14. An alcohol detection system comprises an alcohol detector and an information processing device communicatively connected to each other, wherein,
the alcohol detector includes:
a detecting section that performs alcohol concentration detection in exhaled breath blown by a subject;
a display section; and
a first controller that causes the detecting section to perform the alcohol concentration detection and transmits a detection result of the alcohol concentration detection to the information processing device,
the first controller causes the display section to display an image indicating identification information indicating the alcohol detector during a time from a start of the alcohol concentration detection to transmission of the detection result,
the information processing device comprises an imaging section and a second controller,
the second controller:
causes the imaging section to perform image capture during display of an image including at least the identification information before the detection result is output by the alcohol detector;
acquires the detection result of the alcohol concentration detection after the image section starts image capture; and
determines whether or not the acquired detection result is valid based on the identification information and a captured image captured by the imaging section, and
in determining whether or not the acquired detection result is valid, the second controller determines that the detection result is invalid when the captured image does not include an image matching the identification information.
